(19) United States
(12) Patent Application Publication
(10) Pub. No.: US 2022/0273270 A1
(43) Pub. Date: Sep. 1, 2022

(54) SERVICE METHOD FOR MENSTRUAL CYCLE NOTIFICATION AND PRODUCT RECOMMENDATION THROUGH INTERACTIVE INTERFACE AND APPARATUS THEREFOR

(71) Applicant: RAEL KOREA, INC., Seoul (KR)

(72) Inventors: Ji Eun CHOI, Seongnam (KR); Hyoung Rea KIM, Yongin (KR); Bin Na WON, Seoul (KR)

(21) Appl. No.: 17/625,322

(22) PCT Filed: May 28, 2020
(86) PCT No.: PCT/KR2020/006943

§ 371 Date: Jan. 6, 2022

(30) Foreign Application Priority Data
Nov. 19, 2019 (KR) 10-2019-0148811

(51) Int. Cl. A61B 10/00; A61B 5/00; G06Q 30/06; G06Q 30/02

(52) U.S. Cl. CPC A61B 10/0012; A61B 5/746; G06Q 30/0631; G06Q 30/0255; G06Q 30/0269

(57) ABSTRACT

A service method for menstrual cycle notification and product recommendation through an interactive interface and a service providing apparatus using the same includes: (1) obtaining a user's menstrual information, (2) specifying at least one of a menstrual cycle or the number of sensible menstruation days, (3) providing notification before a set notification date of a next monthly period, and (4) providing a recommended-product list. Even when a user does not know or does not enter her own menstrual information, the user can be informed of statistically predicted menstrual information and recommended with a product customized according to the user's menstrual cycle and menstruation quantity.

### TECHNICAL FIELD

The following description relates to a service method for menstrual cycle notification and product recommendation and a service providing apparatus using the same.

### BACKGROUND ART

A monthly period or menstrual cycle refers to a cycle of physiological changes that occur in women that may be divided into a menstrual phase, a follicular phase, and a luteal phase (secretory phase). An average menstrual cycle length is 28 days, but the actual menstrual cycle length is different for each individual according to various factors.

If a woman can anticipate a monthly period according to her own menstrual cycle, she can adjust her schedule or prepare necessary sanitary products in advance according to the menstrual cycle. Therefore, it is very important for women to know their own menstrual cycles accurately.

Some women write their own menstruation start date in their diary to calculate their menstrual cycle. Software applications that inform a woman of a next menstrual cycle on the basis of a previous menstrual cycle have been also developed and are used by many women. However, the applications are disadvantageous in that a user can only check menstrual cycle information by directly executing the applications. In addition, it is difficult to provide consistent user experiences because methods of use of the applications differ, and it is cumbersome for a user to record her monthly period. In particular, when a user does not know her monthly period or menstrual cycle accurately, the user may be informed of a menstrual cycle incorrectly.

### DISCLOSURE OF INVENTION

### Technical Problem

Provided is a service method for menstrual cycle notification and product recommendation through an interactive interface, which is capable of being easily used by anyone through the interactive interface and providing consistent and intuitive user experiences to all users.

Provided is also a service method for menstrual cycle notification and product recommendation through an interactive interface, which is capable of: (1) providing a user with statistically predicted menstrual information even when the user does not know or does not enter her own menstrual information and (2) recommending a user-customized product according to the user's menstrual cycle and menstruation quantity.

Hereinafter, the present disclosure provides a technical idea of a service method for menstrual cycle notification and product recommendation and a service providing apparatus using the same.

### Solution to Problem

According to one aspect of the present disclosure, a method of providing menstrual cycle notification and a product recommendation service includes: (1) obtaining a user's menstrual information, (2) specifying at least one of a menstrual cycle and the number of sensible menstruation days, (3) providing notification before a set notification date of a next monthly period, and (4) providing a recommended-product list.

According to another aspect of the present disclosure, an apparatus for providing menstrual cycle notification and a product recommendation service includes a memory storing instructions and a processor configured to execute the instructions. Here, the processor is configured to (1) obtain a user's menstrual information, (2) specify at least one of the menstrual cycle or the number of sensible menstruation days, (3) provide notification before a set notification date of a next monthly period, and (4) provide a recommended-product list.

### Advantageous Effects of Invention

The service method for menstrual cycle notification and product recommendation through an interactive interface according to the embodiment of the present disclosure has the following benefits:

1. Anyone can be easily provided with a service in a question-and-answer manner through an interactive interface.

2. Even when a user does not know or does not enter her own menstrual information, the user can be informed of statistically predicted menstrual information.

3. A user can be recommended with user-customized products according to products accessed by the user and the user's menstrual cycle and menstruation quantity.

4. Both menstrual cycle notifications and a product recommendation service can be provided during everyday use of a computing device without having to install a dedicated application.

It should be understood that even effects not described herein are included as effects of the present disclosure when the effects can be expected or anticipated on the basis of the above description of the present disclosure.

### BRIEF DESCRIPTION OF DRAWINGS

FIG. 1 is a diagram illustrating an interface between a computing device and a user in a service method for menstrual cycle notification and product recommendation through an interactive interface according to an embodiment of the present disclosure;

FIG. 2 is a flowchart of a service method for menstrual cycle notification and product recommendation through an interactive interface according to an embodiment of the present disclosure;

FIG. 3 is a diagram illustrating obtaining menstrual information in a service method for menstrual cycle notification and product recommendation through an interactive interface according to an embodiment of the present disclosure;

FIG. 4 is a diagram illustrating obtaining menstrual information in a service method for menstrual cycle notification and product recommendation through an interactive interface according to an embodiment of the present disclosure;

FIG. 5 is a diagram illustrating statistically predicting and specifying a menstrual cycle in a service method for menstrual cycle notification and product recommendation through an interactive interface according to an embodiment of the present disclosure;

FIG. 6 is a diagram illustrating specifying the number of sensible menstruation days in a service method for menstrual cycle notification and product recommendation through an interactive interface according to an embodiment of the present disclosure;

FIG. 7 is a diagram illustrating providing menstrual cycle notification in a service method for menstrual cycle notification and product recommendation through an interactive interface according to an embodiment of the present disclosure;

FIG. 8 is a diagram illustrating providing a recommended-product list in a service method for menstrual cycle notification and product recommendation through an interactive interface according to an embodiment of the present disclosure;

FIG. 9 is a diagram illustrating a recommended-product list configured according to menstruation quantity in a service method for menstrual cycle notification and product recommendation through an interactive interface according to an embodiment of the present disclosure;

FIG. 10 is a diagram illustrating a configuration of a recommended-product list in a service method for menstrual cycle notification and product recommendation through an interactive interface according to an embodiment of the present disclosure; and

FIG. 11 is a diagram illustrating paying for a product included in a recommended-product list in a service method for menstrual cycle notification and product recommendation through an interactive interface according to an embodiment of the present disclosure.

### MODE FOR THE INVENTION

The present disclosure is not limited to embodiments set forth herein and may be embodied in many different forms. These embodiments are provided so that this disclosure will be thorough and complete and will fully convey the concept of the present disclosure to those of ordinary skill in the art.

The terminology used herein is for the purpose of describing the embodiments only and is not intended to be limiting of the present disclosure. The term “comprise” when used herein specifies the presence of a combination of stated features. Therefore, it should be understood that the presence or addition of one or more other features and a combination thereof is not precluded.

In the following description, functions or constructions well-known to technicians in this art are not described in detail when it is determined that they would obscure the present disclosure due to unnecessary detail.

A service method for menstrual cycle notification and product recommendation through an interactive interface according to an embodiment of the present disclosure relates to a method of providing menstrual cycle notification and a product recommendation service according to menstruation quantity through the interactive interface. In general, existing menstrual cycle notification services are provided using a dedicated application installed in a mobile device. In order to receive such a menstrual cycle notification service, a user should execute the dedicated application to directly input and check menstrual information. However, it is difficult to provide consistent and intuitive user experiences with existing service methods because methods of use of applications are different. Moreover, when a user does not input her menstrual information accurately, a menstrual cycle cannot be predicted.

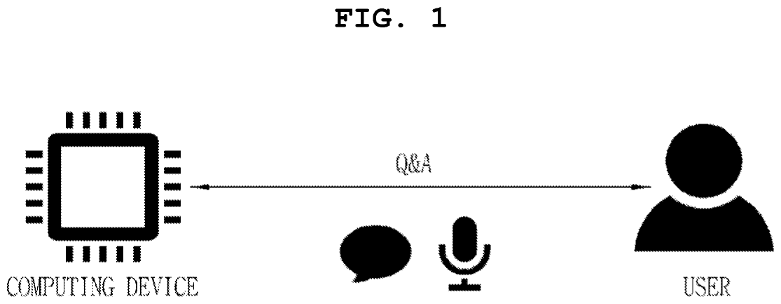

FIG. 1 is a diagram illustrating an interface between a computing device and a user in a service method for menstrual cycle notification and product recommendation through an interactive interface according to an embodiment of the present disclosure. Referring to FIG. 1, in the service method for menstrual cycle notification and product recommendation through an interactive interface according to the embodiment of the present disclosure, a service is provided to a user through the interactive interface. Here, the interactive interface may be implemented through questions and answers by at least one of text or voice. In one embodiment of the present disclosure, a service may be provided through a messenger or assistant application used by a user. That is, the messenger application may provide a service through text communication with a user or the assistant application may provide a service by voice communication with a user. For example, a user may use the messenger or assistant application installed in a mobile device. Alternatively, a speaker device, e.g., a smart speaker in which a voice recognition assistant application is installed, may be used.

Therefore, according to the present disclosure, an easy and intuitive service may be provided in a question-and-answer manner through an interactive interface without having to install a dedicated application for a menstrual cycle notification service by a user. Furthermore, even when a user does not know or does not input her own menstrual information, the user may be informed of her statistically predicted menstrual information. For convenience of explanation, an example of an interactive interface using a messenger application will be described below.

The service method for menstrual cycle notification and product recommendation through an interactive interface according to the embodiment of the present disclosure may be performed by one or more computing devices. In other words, each of operations of the service method may be implemented with one or more instructions executed by a processor of a computing device. Here, the one or more computing devices may include various types of devices with a computing function. In addition, the one or more computing devices may be capable of processing data for providing a service and transmitting data to and receiving data from each other via a network. In one embodiment, the one or more computing devices may be a server and a client which perform a task requested by a user.

For convenience of description, a subject of each operation described below may be omitted. In such cases, it should be understood that each of the operations is performed by a computing device.

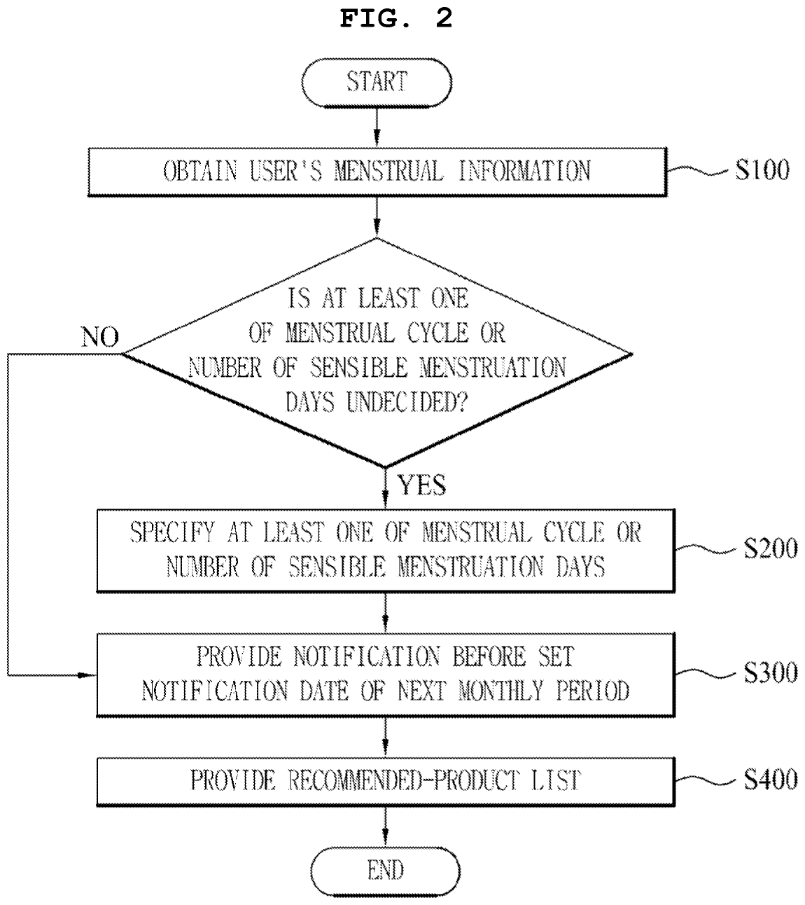

FIG. 2 is a flowchart of a service method for menstrual cycle notification and product recommendation through an interactive interface according to an embodiment of the present disclosure. Referring to FIG. 2, the service method according to an embodiment of the present disclosure includes (1) obtaining a user's menstrual information (S 100), (2) specifying at least one of a menstrual cycle or the number of sensible menstruation days (S200), (3) providing notification before a set notification date of a next monthly period (S300), and (4) providing a recommended-product list (S400). Here, obtaining menstrual information, menstrual cycle notification, and a product recommendation service are provided from a computing device to a user through the interactive interface.

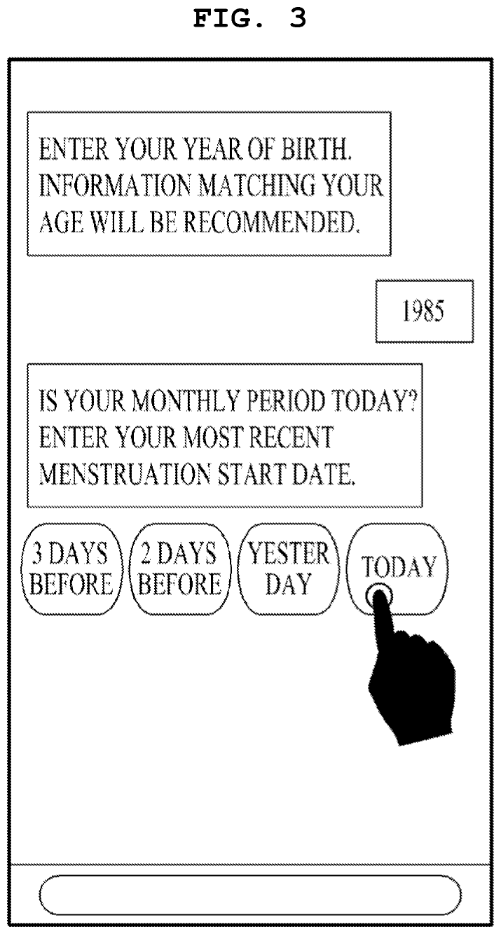

FIG. 3 is a diagram illustrating obtaining menstrual information in a service method for menstrual cycle notification and product recommendation through an interactive interface according to an embodiment of the present disclosure. In operation S100, a user's menstrual information is obtained. Here, the menstrual information is information used to provide the user with menstrual cycle notification and a product recommendation service. The menstrual information includes the year of birth, the most recent menstruation start date, a menstrual cycle, and the number of sensible menstruation days of the user. Referring to FIG. 3, in operation S100, menstrual information may be obtained through questions and answers via the interactive interface. Therefore, in the present disclosure, the user is able to naturally input menstrual information through a conversation without having to additionally learn how to use.

In some embodiments, the obtained menstrual information may be stored in a separate computing device. In this case, the menstrual information may be processed and stored in the form of non-identification information. This is because, even when the user's menstrual information is stored in the form of non-identification information, the user may be provided with a user-customized menstrual cycle notification service according to a value matching data and the menstrual information is statistically predictable. Therefore, personal information may be secured by storing the menstrual information in the form of non-identification information. In one embodiment, the menstrual information may be encrypted and stored.

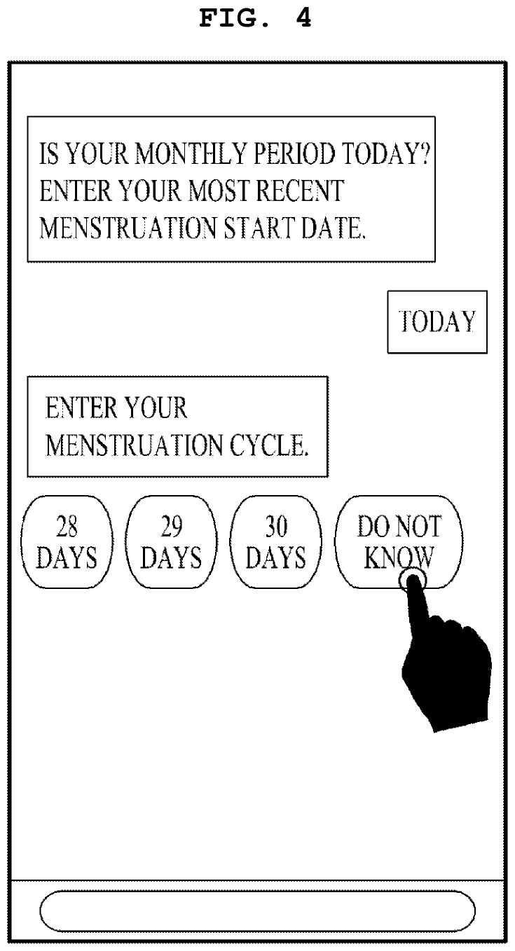

FIG. 4 is a diagram illustrating obtaining menstrual information in a service method for menstrual cycle notification and product recommendation through an interactive interface according to an embodiment of the present disclosure. Referring to FIG. 4, a user may input a specific value as a menstrual cycle. It is necessary to specify the user's menstrual cycle so as to provide the user with a menstrual cycle notification service. However, the menstrual cycle may vary according to various factors and thus it is often difficult for the user to input a correct value. According to the present disclosure, even when the user does not know a precise value and does not specify a menstrual cycle, the menstrual cycle may be statistically predicted and specified. For example, as illustrated in FIG. 4, even when the user does not know her menstrual cycle and thus selects a “don't know” button, the menstrual cycle may be specified.

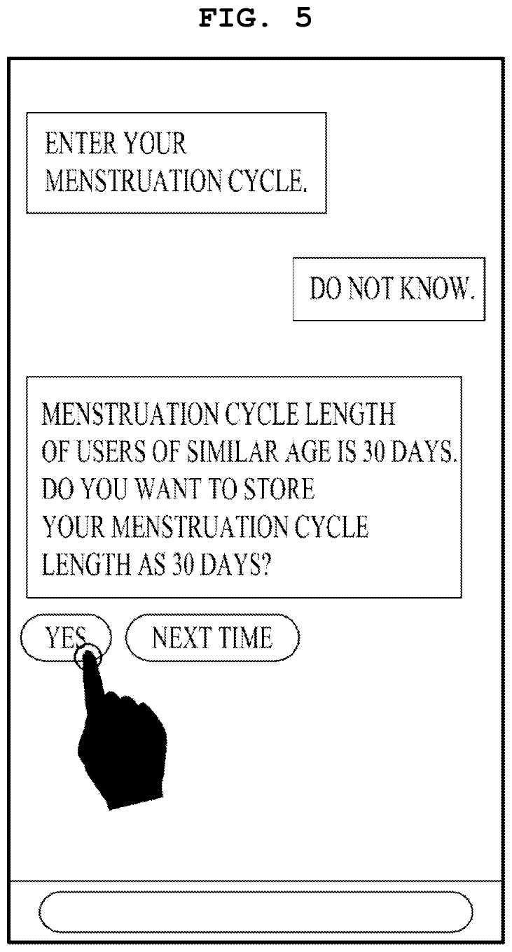

FIG. 5 is a diagram illustrating statistically predicting and specifying a menstrual cycle in a service method for menstrual cycle notification and product recommendation through an interactive interface according to an embodiment of the present disclosure. In operation S200, when the menstrual cycle is undecided, the menstrual cycle is specified using the menstrual information. In detail, in operation S200, the menstrual cycle is specified by referring to a statistic about users of similar age. In operation S200, the menstrual cycle may be specified on the basis of either a specific value or a statistic about the users of similar age according to the number of users of similar age. Here, the users of similar age refer to users corresponding to years of birth within a certain range based on the birth year of the user. For example, when the users of similar age are set as users corresponding to the year of birth of the user ±one year, the number of the users of similar age is a total number of users corresponding to the year of birth of the user±one year.

In some embodiments, when the number of the users of similar age is equal to or less than a reference value, a length of the menstrual cycle is specified to be 28 days, 29 days, or 30 days. When the number of users corresponding to a specific year of birth is much smaller than the reference value, a sufficient number of samples to specify the menstrual cycle are not collected and thus 28 days, 29 days, or 30 days which are an average menstrual cycle length may be specified as the length of the menstrual cycle. However, when the number of users corresponding to the specific year of birth is large, it is more accurate to specify the menstrual cycle on the basis of a statistic according to the age corresponding to the specific year of birth. Therefore, when the number of users of similar age exceeds the reference value, the menstrual cycle is specified as a statistic about menstrual cycles of the users of similar age. In this case, various statistics may be used to specify the menstrual cycle. In some embodiments, a median may be used to specify a representative menstrual cycle while excluding outliers.

Referring to the example of FIG. 5, when the menstrual cycle is undecided, the length of the menstrual cycle may be specified to be 30 days with reference to a statistic about users of similar age. Therefore, according to the present disclosure, the user's menstrual cycle may be statistically specified even when the user fails to input a correct menstrual cycle.

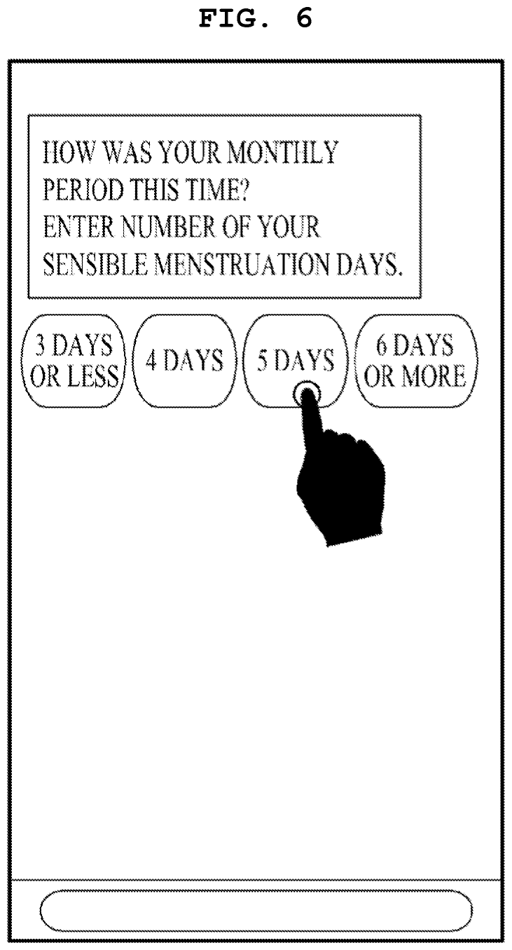

FIG. 6 is a diagram illustrating specifying the number of sensible menstruation days in a service method for menstrual cycle notification and product recommendation through an interactive interface according to an embodiment of the present disclosure. In the present disclosure, the number of sensible menstruation days refers to the number of days that the user actually feels that she noticeably menstruated and is used to provide a product recommendation service. Therefore, it is necessary to specify the number of sensible menstruation days so as to provide the product recommendation service to the user. However, similar to a menstrual cycle, the number of sensible menstruation days may vary according to various factors and thus it is often difficult for the user to input a correct value. In addition, the number of sensible menstruation days is information that can be input after the end of menstruation and thus it would be cumbersome for the user who has completed menstruation to calculate the number of sensible menstruation days accurately. According to the present disclosure, even when the user does not know a correct value and thus cannot specify the number of sensible menstruation days, the number of sensible menstruation days may be statistically predicted and specified.

In operation S200, when the number of sensible menstruation days is undecided, the number of sensible menstruation days is specified using the menstrual information. Specifically, in operation S200, the number of sensible menstruation days is specified by referring to a statistic about users of similar age. In operation S200, the number of sensible menstruation days may be specified on the basis of either a specific value or the statistic about the users of similar age according to the number of users of similar age. When the number of users of similar age is less than or equal to a reference value, the number of sensible menstruation days is specified to be any one of three, four, and five. When the number of users of similar age exceeds the reference value, the number of sensible menstruation days is specified to be a statistic about the number of sensible menstruation days of the users of similar age. In this case, various statistics may be used to specify the number of sensible menstruation days. In one embodiment, a median may be used to specify a general number of menstruation days while excluding outliers.

The reference value is a value used as a criterion for determining how to specify the menstrual cycle and the number of sensible menstruation days according to the number of users of similar age. The reference value may be variously determined according to the number of users. For example, the reference value may be set to a small value when the number of users of similar age is small and may be set to a large value when the number of users of similar age is large.

In summary, in operation S200, when at least one of the menstrual cycle or the number of sensible menstruation days is undecided, at least one of the menstrual cycle or the number of sensible menstruation days is specified using the menstrual information. The more data regarding menstrual cycles and the numbers of menstruation days of users is accumulated, the more accurately the menstrual cycle and the number of sensible menstruation days may be predicted according to age. Therefore, according to the present disclosure, a menstrual cycle and the number of sensible menstruation days may be predicted more accurately as data regarding users accumulates and thus a service may be provided to a user who does not accurately know her own menstrual cycle and number of menstruation days.

Meanwhile, there are cases in which a user directly inputs a menstrual cycle and the number of sensible menstruation days. In this case, the menstrual cycle and the number of sensible menstruation days are specified and thus operation S300 may be performed after operation S100. However, in some embodiments, when it is determined that a value input by the user is not correct, a statistically specified menstrual cycle and the number of sensible menstruation days may be used.

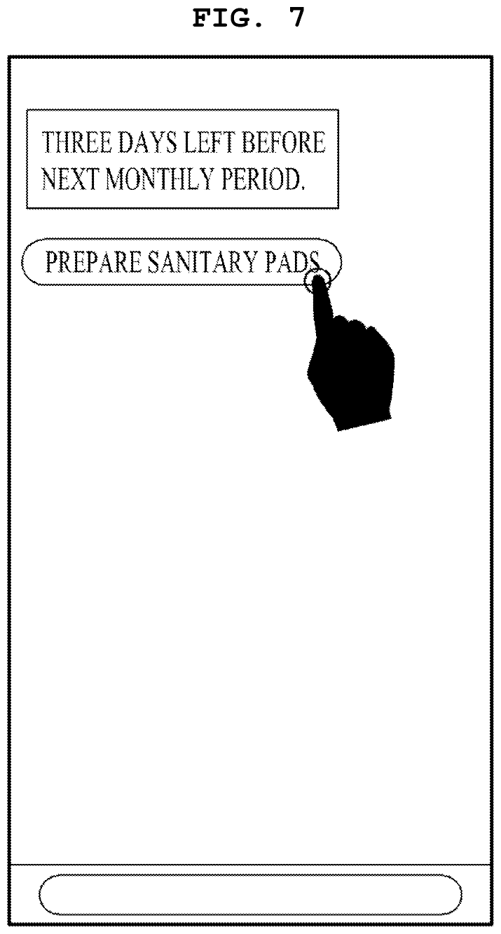

FIG. 7 is a diagram illustrating providing menstrual cycle notification in a service method for menstrual cycle notification and product recommendation through an interactive interface according to an embodiment of the present disclosure. Referring to FIG. 7, in operation S300, a notification is provided using the most recent menstruation start date and a menstrual cycle before a set notification date of a next monthly period. That is, the notification is provided when the following equation is satisfied. Here, the set notification date may be set variously.

today's date=most recent menstruation start date+menstrual cycle−set notification date [Equation 1]

In the example illustrated in FIG. 7, the set notification date is three days, and a menstrual cycle notification service is provided from a computing device to a user as Equation 1 is satisfied. According to the present disclosure, a service may be provided through a messenger or assistant application mainly used by a user without having to install a dedicated application for a menstrual cycle notification service. Therefore, the menstrual cycle notification service may be naturally provided to the user during everyday use of the computing device.

In addition, referring to FIG. 7, according to the present disclosure, the user may be recommended to prepare sanitary products while being provided with the notification before a menstruation start date according to the set notification date. Therefore, the user may prepare sanitary products in advance before the menstruation start date, thereby preventing a situation in which no sanitary product is available on the menstruation start date.

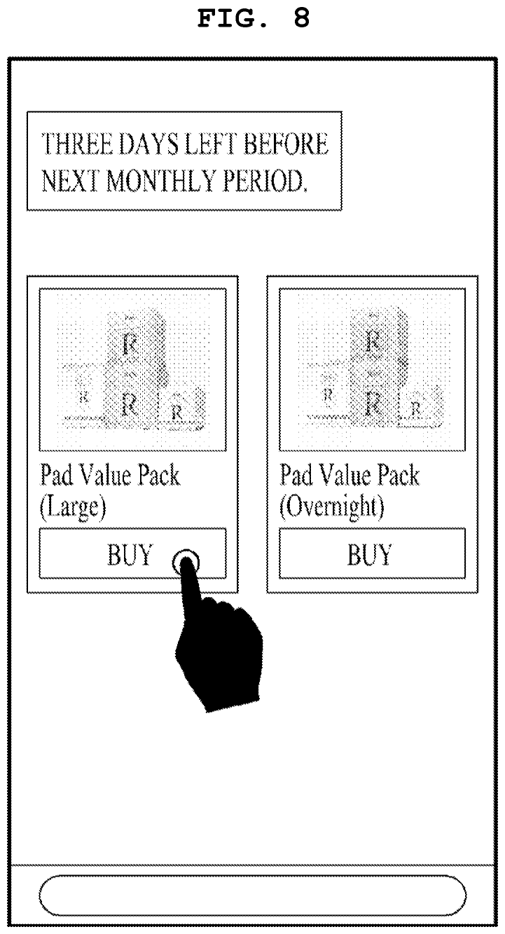

FIG. 8 is a diagram illustrating providing a recommended-product list in a service method for menstrual cycle notification and product recommendation through an interactive interface according to an embodiment of the present disclosure. Referring to FIG. 8, in operation S400, a recommended-product list is provided using the menstrual information and preferred-product information. According to the present disclosure, the recommended-product list may be provided that is customized for the user according to the user's menstruation quantity.

For convenience of explanation, an example of a recommended-product list including only sanitary pads will described below. Among various sanitary products, sanitary pads may be largely classified into light sanitary pads, moderate sanitary pads, heavy sanitary pads, and overnight sanitary pads according to menstruation quantity. Therefore, the user should use sanitary pads according to her own menstruation quantity.

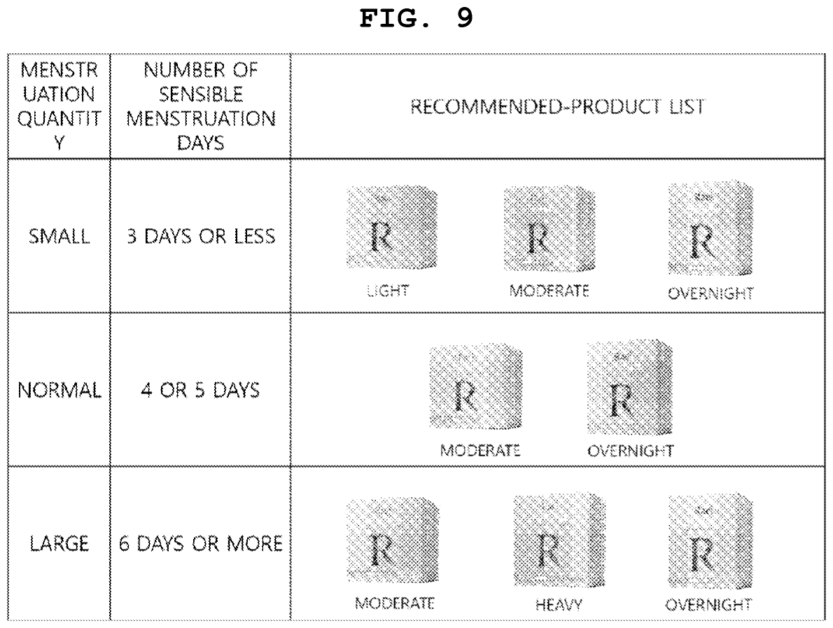

FIG. 9 is a diagram illustrating a recommended-product list configured according to menstruation quantity in a service method for menstrual cycle notification and product recommendation through an interactive interface according to an embodiment of the present disclosure. In operation S400, menstruation quantity is divided into several categories and a recommended-product list is created for each of the categories according to menstruation quantity. For example, as illustrated in FIG. 9, when menstruation quantity is categorized into “small,” “normal,” and “large,” a list of products recommended according to menstruation quantity may be created for each of the categories.

In the present disclosure, because it is difficult for a user to directly specify menstruation quantity, the menstruation quantity is estimated according to the number of sensible menstruation days. The number of sensible menstruation days is an important parameter for estimation of menstruation quantity and is proportional to the menstruation quantity. That is, the menstruation quantity decreases as the number of sensible menstruation days decreases and increases as the number of sensible menstruation days increases. Therefore, in operation S400, the menstruation quantity is estimated by the number of sensible menstruation days and products are recommended according to the number of sensible menstruation days.

Referring to the example of FIG. 9, in operation S400, when the number of sensible menstruation days is three or less, a recommended-product list including light, moderate, and overnight products is created. When the number of sensible menstruation days is three or less, the menstruation quantity may be estimated to be small. Thus, the recommended-product list includes light, moderate, and overnight products suitable for users with small menstruation quantity.

In operation S400, when the number of sensible menstruation days is four or five, a recommended-product list including moderate and overnight products is created. When the number of sensible menstruation days is four or five, the menstruation quantity may be estimated to be medium. Therefore, the recommended-product list includes moderate and overnight products suitable for users with normal menstruation quantity.

In operation S400, when the number of sensible menstruation days is six or more, a recommended-product list including moderate, heavy and overnight products is created. When the number of sensible menstruation days is six or more, the menstruation quantity may be estimated to be large. Thus, the recommended-product list includes moderate, heavy, and overnight products suitable for users with large menstruation quantity.

In the example of FIG. 9, menstruation quantity is categorized into “small,” “normal,” and “large,” which respectively match three days or less, four or five days, and six days or more as the number of sensible menstruation days. In some embodiments, menstruation quantity may be divided into larger or smaller categories, and the number of sensible menstruation days for estimation of the menstruation quantity may also be divided and matched with the menstruation quantity accordingly.

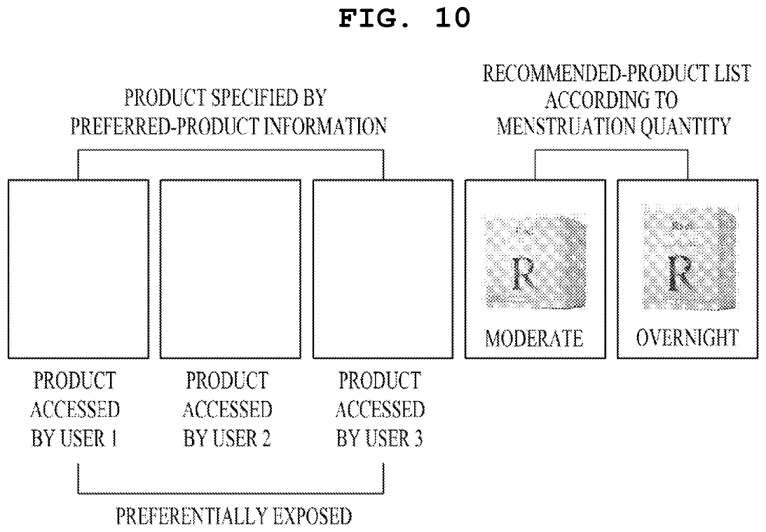

FIG. 10 is a diagram illustrating a configuration of a recommended-product list in a service method for menstrual cycle notification and product recommendation through an interactive interface according to an embodiment of the present disclosure. Referring to FIG. 10, in operation S400, a product specified using preferred-product information is preferentially exposed in a recommended-product list. Here, the preferred-product information includes information regarding products accessed by a user. In some embodiments, products accessed within a predetermined time period may be filtered as the products accessed by the user. Therefore, products recently accessed by the user may be preferentially specified using the preferred-product information. Here, the predetermined time period may be variously set.

In some embodiments, in operation S400, only a predetermined number of preferred products are exposed in the recommended-product list. In the example of FIG. 10, it is assumed that the number of products included in the recommended-product list is five and a predetermined number of preferred products is three. In this case, in operation S400, only three products among products specified by the preferred-product information are exposed in the recommended-product list. In other words, in operation S400, a “product 1,” a “product 2,” and a “product 3” among products recently accessed by a user are preferentially exposed in the recommended-product list. The other two products are exposed in the recommended-product list according to menstruation quantity. Therefore, the user may be recommended preferentially with a product recently accessed by the user in the recommended-product list and then be recommended with a product according to her own menstruation quantity.

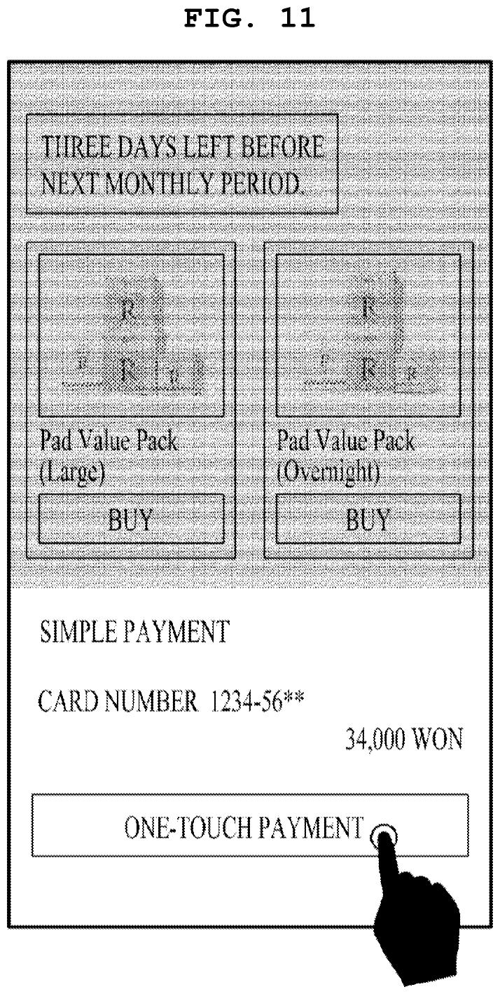

FIG. 11 is a diagram illustrating paying for a product included in a recommended product list in a service method for menstrual cycle notification and product recommendation through an interactive interface according to an embodiment of the present disclosure. Referring to FIG. 11, a user may immediately pay for a product included in a recommended-product list. That is, the user may be provided with a menstrual cycle notification service and a product recommendation service and may continuously and immediately proceed with payment for a product. Therefore, according to the present disclosure, all services related to menstruation, including menstrual cycle notification and purchase of menstrual products, may be provided through a user-friendly interactive interface.

An apparatus for providing menstrual cycle notification and a product recommendation service through an interactive interface according to another embodiment of the present disclosure includes a memory storing instructions and a processor configured to execute the instructions. The processor performs: (1) obtaining a user's menstrual information (S100), (2) specifying at least one of a menstrual cycle or the number of sensible menstruation days (S200), (3) providing notification before a set notification date of a next monthly period (S300), and (4) providing a recommended product list (S400). That is, the apparatus for providing menstrual cycle notification and a product recommendation service through an interactive interface according to an embodiment of the present disclosure is an apparatus which performs a service method for menstrual cycle notification and product recommendation through an interactive interface.

A server for providing menstrual cycle notification and a product recommendation service through an interactive interface according to another embodiment of the present disclosure includes a receiver, a specifier, a notification provider, and a list provider. The receiver receives a user's menstrual information including the year of birth, the most recent menstruation start date, a menstrual cycle, and the number of sensible menstruation days of the user. When at least one of the menstrual cycle or the number of sensible menstruation days received by the receiver is undecided, the specifier specifies at least one of the menstrual cycle or the number of sensible menstruation days on the basis of the menstrual information. The notification provider provides notification to a user device using the most recent menstruation start date and the menstrual cycle before a set notification date of a next monthly period. The list provider provides a recommended-product list to the user device using the menstrual information and preferred-product information. Here, the user device is a device used by the user. That is, the server for providing menstrual cycle notification and a product recommendation service through an interactive interface according to the embodiment of the present disclosure is a server which provides a user device with a service method for menstrual cycle notification and product recommendation through an interactive interface.

A computer program stored in a computer-readable recording medium according to another embodiment of the present disclosure may include instructions for performing a service method for menstrual cycle notification and product recommendation through an interactive interface. That is, when the computer program including the instructions is loaded into the memory, the processor executes the loaded instructions to perform the service method for menstrual cycle notification and product recommendation through an interactive interface.

The matters mentioned in the above description of the present disclosure are merely examples and thus may be variously modified and implemented by technicians in the art. Therefore, the scope of the present disclosure is not limited by the description and expression of the embodiments set forth herein. A system for providing a menstrual cycle notification and a product recommendation service through an interactive interface may include a server including a user information database and a storage medium, a messenger server providing and an interactive messenger service with the user through a messenger application, a computing device on which the messenger application is installed, and an ecommerce server providing a payment transaction service. The server is connectable to the Internet and can communicate with the computing device, the messenger server, and the ecommerce server over the Internet. The computing device may be a mobile electronic communications device, smartphone, desktop computer, laptop computer and tablet computer, and the like. The messenger server may be Kakao talk, What's App, or other messenger services. The payment transaction service of the ecommerce server may be provided by the server.

1. A method of providing menstrual cycle notification and a product recommendation service through an interactive interface using a computing device, the method comprising:
(1) obtaining a user's menstrual information including a year of birth, a most recent menstruation start date, a menstrual cycle length, and a number of sensible menstruation days of the user;
(2) when at least one of the menstrual cycle length or the number of sensible menstruation days obtained in operation (1) is undecided, specifying at least one of the menstrual cycle length or the number of sensible menstruation days using the menstrual information;
(3) providing a notification using the most recent menstruation start date and the menstrual cycle length before a set notification date of a next monthly period; and
(4) providing a recommended-product list using the menstrual information and preferred-product information, wherein a service is provided via the interactive interface,
wherein, after the user's menstruation is completed, the number of sensible menstruation days is information is obtained and is used to provide the recommended-product list in operation (4),
wherein specifying the number of sensible menstruation days in operation (2) comprises referring to a statistic about users of similar age,
wherein specifying the number of sensible menstruation days in operation (2) comprises using either a specific value or a statistic about the users of similar age according to the number of users of similar age,
wherein the number of sensible menstruation days is specified to be any one of three, four, and five days when the number of the users of similar age is less than or equal to a reference value and is specified to be a statistic about the number of sensible menstruation days of the users of similar age when the number of users of similar age is greater than the reference value,
wherein providing the recommended-product list in operation (4) comprises creating the recommended-product list with different configuration of products according to the number of sensible menstruation days,
wherein providing the recommended-product list in operation (4) comprises showing products specified by preferred-product information preferentially in the recommended-product list, the preferred-product information including information regarding products accessed by the user, and
wherein providing the recommended-product list in operation (4) comprises showing only a predetermined number of preferred products in the recommended-product list among the products specified by the preferred product information.

2. The method of claim 1, wherein specifying the menstrual cycle length in operation (2) comprises referring to a statistic about users of similar age.

3. The method of claim 1, wherein specifying the menstrual cycle length in operation (2) comprises using either a specific value or a statistic about the users of similar age according to the number of users of similar age.

4. The method of claim 3, wherein the length of the menstrual cycle is specified to be any one of 28 days, 29 days, and 30 days when the number of the users of similar age is less than or equal to a reference value and is specified to be a statistic about menstrual cycles of the users of similar age when the number of users of similar age is greater than the reference value.

5-8. (canceled)

9. The method of claim 1, wherein providing the recommended-product list in operation (4) comprises dividing menstruation quantity into several categories and creating the recommended-product list for each of the categories according to the menstruation quantity using the number of sensible menstruation days.

10. The method of claim 1, wherein providing the recommended-product list in operation (4) comprises:
creating a first recommended-product list including light, moderate, and overnight products when the number of sensible menstruation days is three or less, creating a second recommended-product list including moderate and overnight products when the number of sensible menstruation days is four or five, and creating a third recommended-product list including moderate, heavy and overnight products when the number of sensible menstruation days is six or more; and
selecting the recommended-product list from among the first, second, and third recommended-product lists.

11-12. (canceled)

13. The method of claim 1, wherein the menstrual information is processed and stored in the form of non-identification information.

14. The method of claim 1, wherein the interactive interface is implemented in a question-and-answer manner using at least one of text or voice.

15. An apparatus for providing menstrual cycle notification and a product recommendation service through an interactive interface, the apparatus comprising:
a memory storing instructions; and
a processor configured to execute the instructions, wherein the processor is configured to:
(1) obtain a user's menstrual information including a year of birth, a most recent menstruation start date, a menstrual cycle length, and a number of sensible menstruation days of the user;
(2) when at least one of the menstrual cycle length or the number of sensible menstruation days obtained in operation (1) is undecided, specify at least one of the menstrual cycle length or the number of sensible menstruation days using the menstrual information;
(3) provide a notification using the most recent menstruation start date and the menstrual cycle length before a set notification date of a next monthly period; and
(4) provide a recommended-product list using the menstrual information and preferred-product information, and a service is provided via the interactive interface,
wherein, after the user's menstruation is completed, the number of sensible menstruation days is information is obtained and is used to provide the recommended-product list in operation (4),
wherein specifying the number of sensible menstruation days in operation (2) comprises referring to a statistic about users of similar age,
wherein specifying the number of sensible menstruation days in operation (2) comprises using either a specific value or a statistic about the users of similar age according to the number of users of similar age,
wherein the number of sensible menstruation days is specified to be any one of three, four, and five days when the number of the users of similar age is less than or equal to a reference value and is specified to be a statistic about the number of sensible menstruation days of the users of similar age when the number of users of similar age is greater than the reference value,
wherein providing the recommended-product list in operation (4) comprises creating the recommended-product list with different configuration of products according to the number of sensible menstruation days,
wherein providing the recommended-product list in operation (4) comprises showing products specified by preferred-product information preferentially in the recommended-product list, the preferred-product information including information regarding products accessed by the user, and
wherein providing the recommended-product list in operation (4) comprises showing only a predetermined number of preferred products in the recommended-product list among the products specified by the preferred product information.

16. A server for providing menstrual cycle notification and a product recommendation service through an interactive interface, the server comprising:
a receiver configured to receive a user's menstrual information including a year of birth, a most recent menstruation start date, a menstrual cycle length, and a number of sensible menstruation days of the user;
a specifier configured to specify at least one of the menstrual cycle length or the number of sensible menstruation days using the menstrual information when at least one of the menstrual cycle length or the number of sensible menstruation days received by the receiver is undecided;
a notification provider configured to provide a user device with a notification using the most recent menstruation start date and the menstrual cycle length before a set notification date of a next monthly period; and
a list provider configured to provide a recommended-product list to the user device using the menstrual information and preferred-product information,
wherein a service is provided via the interactive interface,
wherein, after the user's menstruation is completed, the number of sensible menstruation days is information is obtained and is used to provide a recommended-product list in the list provider,
wherein the specifier specifies the number of sensible menstruation days by referring to a statistic about users of similar age,
wherein the specifier specifies the number of sensible menstruation days using either a specific value or a statistic about the users of similar age according to the number of users of similar age,
wherein the number of sensible menstruation days is specified to be any one of three, four, and five when the number of the users of similar age is less than or equal to a reference value and is specified to be a statistic about the number of sensible menstruation days of the users of similar age when the number of users of similar age is greater than the reference value,
wherein the list provider creates a recommended-product list with different configuration of products according to the number of sensible menstruation days,
wherein the list provider shows products specified by preferred-product information preferentially in the recommended-product list, the preferred-product information including information regarding products accessed by the user, and
wherein the list provider shows only a predetermined number of preferred products in the recommended-product list among the products specified by the preferred product information.

17. A computer program stored in a computer-readable recording medium, the computer program comprising instructions configured to:
(1) obtain a user's menstrual information including a year of birth, the most recent menstruation start date, a menstrual cycle length, and the number of sensible menstruation days of a user;
(2) when at least one of the menstrual cycle length or the number of sensible menstruation days obtained in operation (1) is undecided, specify at least one of the menstrual cycle length or the number of sensible menstruation days using the menstrual information;
(3) provide a notification using the most recent menstruation start date and the menstrual cycle length before a set notification date of a next monthly period; and
(4) provide a recommended-product list using the menstrual information and preferred-product information, and
wherein a service is provided via an interactive interface,
wherein, after the user's menstruation is completed, the number of sensible menstruation days is information is obtained and is used to provide the recommended-product list in operation (4), wherein specifying the number of sensible menstruation days in operation (2) comprises referring to a statistic about users of similar age,
wherein specifying the number of sensible menstruation days in operation (2) comprises using either a specific value or a statistic about the users of similar age according to the number of users of similar age,
wherein the number of sensible menstruation days is specified to be any one of three, four, and five days when the number of the users of similar age is less than or equal to a reference value and is specified to be a statistic about the number of sensible menstruation days of the users of similar age when the number of users of similar age is greater than the reference value,
wherein providing the recommended-product list in operation (4) comprises creating the recommended-product list with different configuration of products according to the number of sensible menstruation days,
wherein providing the recommended-product list in operation (4) comprises showing products specified by preferred-product information preferentially in the recommended-product list, the preferred-product information including information regarding products accessed by the user, and
wherein providing the recommended-product list in operation (4) comprises showing only a predetermined number of preferred products in the recommended-product list among the products specified by the preferred product information.
